(19) United States
(12) Patent Application Publication
(10) Pub. No.: US 2012/0029290 A1
(43) Pub. Date: Feb. 2, 2012

(54) ENDOSCOPE APPARATUS AND OPTICAL ADAPTER USED IN THE ENDOSCOPE APPARATUS

(75) Inventor: Yoshikazu Nishijima, Uenohara-shi (JP)

(73) Assignee: Olympus Corporation, Tokyo (JP)

(21) Appl. No.: 13/269,122

(22) Filed: Oct. 7, 2011

(30) Foreign Application Priority Data
Dec. 24, 2009 (JP) 2009-293080

(51) Int. Cl. A61B 1/12

(52) U.S. Cl. 600/156

(57) ABSTRACT

An endoscope apparatus is provided that includes a plurality of kinds of optical adapters that have an illuminating window and an observation window, and an endoscope insertion portion that has a distal end portion to which the plurality of kinds of optical adapters can be detachably attached. A fluid spout that ejects fluid at least at the observation window, and a fluid inlet whose arrangement position is common among the plurality of kinds of optical adapters and that is connected to and communicates with a fluid supply port for supplying the fluid that is arranged on a distal end face of the distal end portion are provided in the optical adapters. A fluid supply channel that causes the fluid inlet and the fluid spout to communicate and in which the fluid flows and the fluid spout are configured differently according to the kind of the optical adapter.

### BACKGROUND OF THE INVENTION

1. Field of the Invention The present invention relates to an endoscope apparatus and an optical adapter.

2. Description of the Related Art

In recent years, endoscopes have been widely used in the medical and industrial fields.

An endoscope used in the medical field can observe an organ inside a body cavity that is a site to be examined by inserting an elongated insertion portion into the body cavity. As necessary, the endoscope can perform various kinds of treatment using a treatment instrument that is inserted inside a treatment instrument insertion channel.

Further, an endoscope used in the industrial field, by inserting the elongated insertion portion inside a jet engine or pipes of a factory or the like as a site to be examined, can be used for observation to check for the presence of flaws or corrosion or the like at the site to be examined and to perform various kinds of repairs.

An objective optical system, an image pickup section that has an image pickup device such as a CCD, and an illumination section that illuminates a site to be examined and the like are provided in a distal end portion of an insertion portion of an endoscope. A configuration is also known in which the objective optical system and the illumination section are provided in a detachable optical adapter at the distal end portion of the insertion portion.

Various types of optical adapters are available. Examples thereof include a single lens front-view type, a single lens side-view type, a binocular front-view type, and a binocular side-view type. The various types of optical adapters are appropriately used according to an observation target and an intended use.

Generally, to improve an observation performance of an endoscope, cleaning is performed that wipes off dirt that is adhered to an observation window and the like provided on a distal end face of a distal end portion of an insertion portion of the endoscope by ejecting fluid towards the observation window and the like through a spout that is similarly provided in the distal end face.

In an endoscope used in the industrial field also, it is necessary to clean an observation window and the like using fluid to improve an observation performance, and in particular, there is a strong need with respect to endoscope apparatuses to which the aforementioned plurality of kinds of optical adapters can be detachably attached.

A conventional technology relating to optical adapters in which a conduit for supplying fluid is provided inside the aforementioned types of optical adapters includes, for example, an endoscope apparatus described in Japanese Patent Application Laid-Open Publication No. 2007-195798.

The endoscope apparatus described in Japanese Patent Application Laid-Open Publication No. 2007-195798 has a water storage section that stores water inside a lens barrel part that is included in a distal end adapter, and includes a linear adapter-side conduit that is provided in substantially the insertion axis direction. The endoscope apparatus is configured so as to dissipate heat of the lens barrel part that is transmitted from LED chips inside the distal end portion of the insertion portion by adopting a configuration such that water is supplied to the adapter-side conduit from a water supply tube which communicates with the adapter-side conduit, and inside the endoscope insertion portion and stored in the water storage section, and an excessive amount of water flows to a drainage channel.

### SUMMARY OF THE INVENTION

An endoscope apparatus according to one aspect of the present invention includes a plurality of kinds of optical adapters having an illuminating window that radiates an illuminating light and an observation window through which a subject can be viewed, and an endoscope insertion portion having a distal end portion to which the plurality of optical adapters can be detachably attached, wherein: a fluid spout that ejects fluid at least at the observation window, and fluid inlet whose arrangement position is common among the plurality of kinds of optical adapters and that is connected to and communicates with a fluid supply port for supplying the fluid that is arranged on a distal end face of the distal end portion of the endoscope insertion portion are provided in each of the optical adapters; and a fluid supply channel that causes the fluid inlet and the fluid spout to communicate and in which the fluid flows and the fluid spout are configured differently according to a kind of the optical adapter.

Further, according to another aspect of the present invention, there is provided a plurality of kinds of optical adapters that can be detachably attached to a distal end portion of an endoscope insertion portion and that have an illuminating window that radiates an illuminating light and an observation window through which a subject can be viewed, wherein: a fluid spout that ejects fluid at least at the observation window, and a fluid inlet that is connected to and communicates with a fluid supply port for supplying the fluid that is arranged on a distal end face of the distal end portion of the endoscope insertion portion are provided in each of the optical adapters; and a fluid supply channel that causes the fluid inlet and the fluid spout to communicate and in which the fluid flows and the fluid spout are configured differently according to a kind of the optical adapter.

### BRIEF DESCRIPTION OF THE DRAWINGS

FIG. 1 is a perspective view that shows a configuration of an endoscope apparatus according to Embodiment 1 of the present invention;

FIG. 2 is a perspective view that shows a configuration of a distal end portion of an endoscope insertion portion shown in FIG. 1;

FIG. 3 is a front view that shows a configuration of a distal end face of the distal end portion shown in FIG. 2;

FIG. 4 is a perspective view of a single lens front-view optical adapter shown in FIG. 1, as viewed from a side that is attached to the insertion portion;

FIG. 5 is a side view of the single lens front-view optical adapter shown in FIG. 4;

FIG. 6 is a front view that shows a configuration of a distal end face of the single lens front-view optical adapter shown in FIG. 5;

FIG. 7 is a plan view of the single lens front-view optical adapter shown in FIG. 5, as viewed from a side that is attached to an insertion portion;

FIG. 8 is a view that shows a distal end face side of the endoscope apparatus to which an optical adapter shown in FIG. 1 is attached;

FIG. 9 is a sectional view along a line A-A in FIG. 8;

FIG. 10 is a sectional view that shows a modification example of the single lens front-view optical adapter according to Embodiment 1, which shows a configuration of an endoscope apparatus to which the optical adapter is attached;

FIG. 11 is a perspective view that shows a configuration of the endoscope apparatus shown in FIG. 10;

FIG. 12 is a perspective view that shows a configuration of an endoscope apparatus according to Embodiment 2 of the present invention;

FIG. 13 is a perspective view of a single lens side-view optical adapter shown in FIG. 12, as viewed from a side that is attached to an insertion portion;

FIG. 14 is a side view of the single lens side-view optical adapter shown in FIG. 13;

FIG. 15 is a front view that shows a configuration of a distal end face of the single lens side-view optical adapter shown in FIG. 13;

FIG. 16 is a plan view of the single lens side-view optical adapter shown in FIG. 13, as viewed from a side that is attached to an insertion portion;

FIG. 17 is a view that shows a distal end face side of an endoscope apparatus to which the optical adapter shown in FIG. 13 is attached;

FIG. 18 is a sectional view along a line B-B in FIG. 17;

FIG. 19 is a perspective view that shows a configuration of an endoscope apparatus according to Embodiment 3 of the present invention;

FIG. 20 is a perspective view of a binocular front-view optical adapter shown in FIG. 19, as viewed from a side that is attached to an insertion portion;

FIG. 21 is a side view of the binocular front-view optical adapter shown in FIG. 20;

FIG. 22 is a front view that shows a configuration of a distal end face of the binocular front-view optical adapter shown in FIG. 20;

FIG. 23 is a plan view of the binocular front-view optical adapter shown in FIG. 20, as viewed from a side that is attached to an insertion portion;

FIG. 24 is a view that shows a distal end face side of an endoscope apparatus to which the binocular front-view optical adapter shown in FIG. 20 is attached;

FIG. 25 is a sectional view along a line C-C in FIG. 24;

FIG. 26 is a sectional view along a line D-D in FIG. 24;

FIG. 27 is a perspective view that shows a configuration of an endoscope apparatus according to Embodiment 4 of the present invention;

FIG. 28 is a perspective view of the endoscope apparatus shown in FIG. 27, as viewed from a diagonally downward direction in the figure;

FIG. 29 is a perspective view of a binocular side-view optical adapter shown in FIG. 28, as viewed from a side that is attached to an insertion portion;

FIG. 30 is a side view of the binocular side-view optical adapter shown in FIG. 28;

FIG. 31 is a front view that shows a configuration of a distal end face of the binocular side-view optical adapter shown in FIG. 28;

FIG. 32 is a plan view of the binocular side-view optical adapter shown in FIG. 28, as viewed from a side that is attached to an insertion portion;

FIG. 33 is a view that shows a distal end face side of the endoscope apparatus to which an optical adapter shown in FIG. 27 is attached;

FIG. 34 is a sectional view along a line E-E in FIG. 33;

FIG. 35 is a sectional view along a line F-F in FIG. 33;

FIG. 36 is a sectional view along a line G-G in FIG. 34; and

FIG. 37 is a sectional view along a line H-H in FIG. 35.

### DETAILED DESCRIPTION OF THE PREFERRED EMBODIMENTS

Embodiments of the present invention are described in detail hereunder while referring to the accompanying drawings.

### Embodiment 1

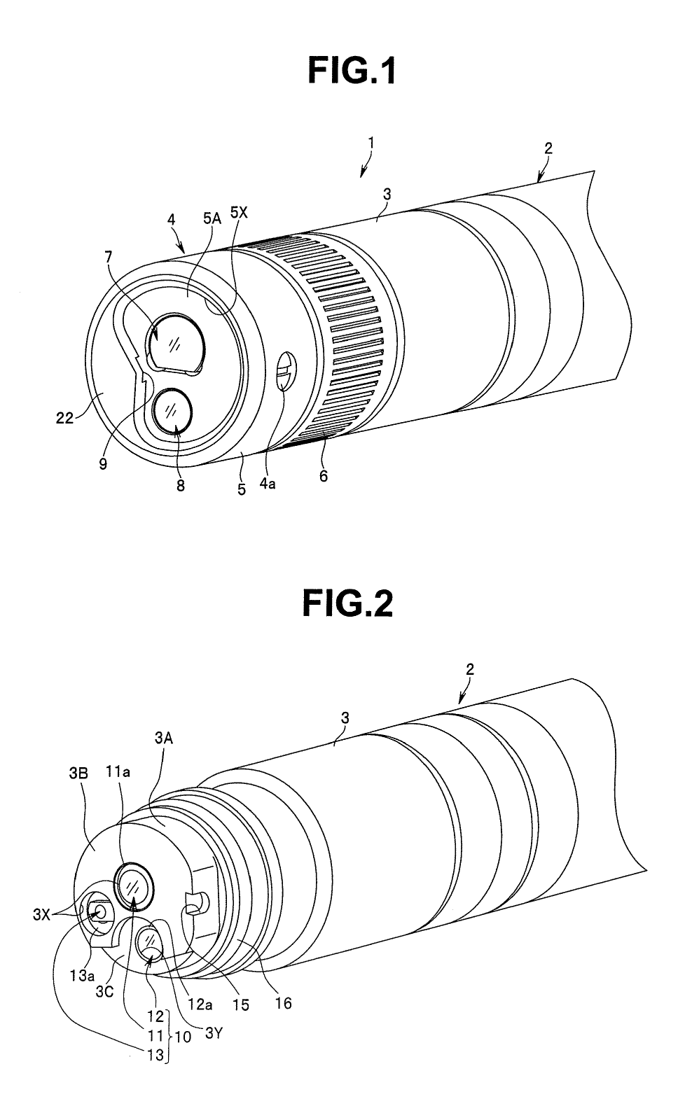

FIG. 1 to FIG. 9 illustrate Embodiment 1 of an endoscope apparatus according to the present invention.

As shown in FIG. 1, an endoscope apparatus 1 of the present embodiment includes a flexible, elongated endoscope insertion portion (hereunder, abbreviated to insertion portion) 2 that is inserted into a body cavity or a structure, and a plurality of kinds of optical adapters 4 that can be detachably attached to a distal end portion 3 provided on a distal end side of the insertion portion 2.

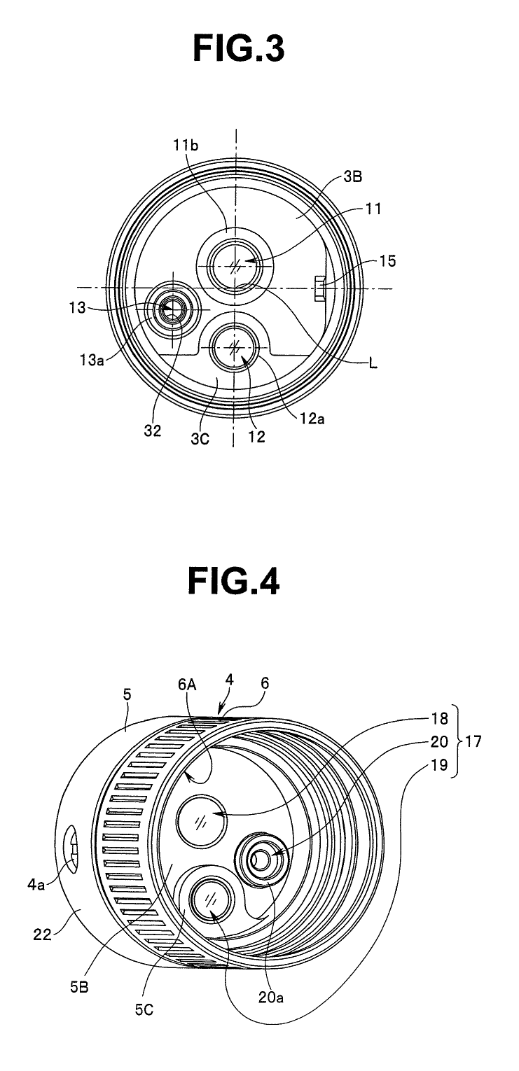

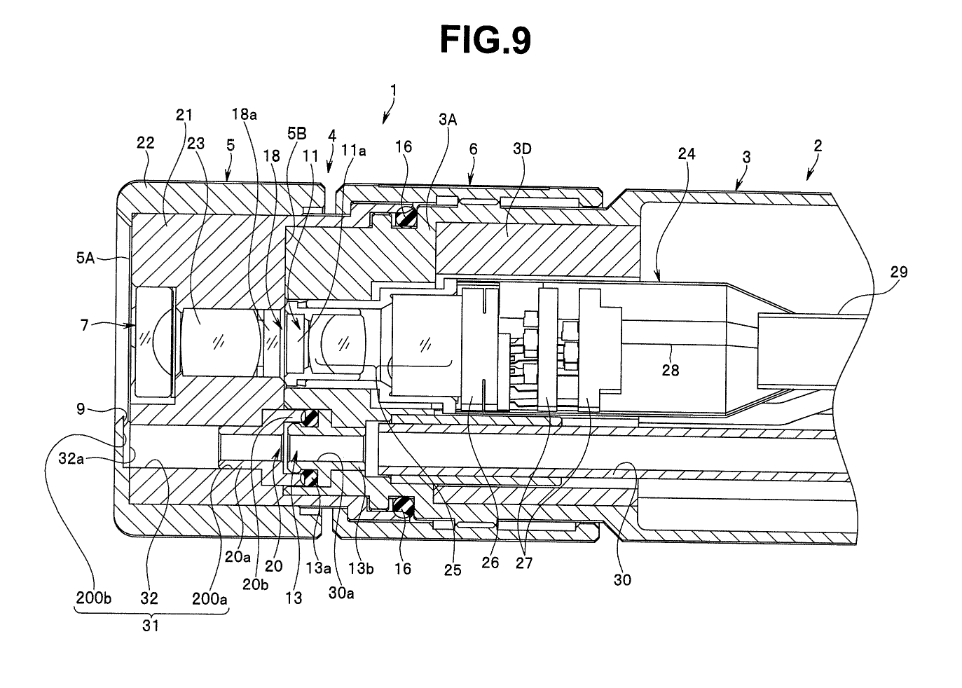

First, a configuration of the distal end portion 3 of the insertion portion 2 will be described using FIG. 2, FIG. 3 and FIG. 9.

As shown in FIG. 2, a protruding portion 3A that protrudes in an insertion direction is provided on a distal end side of the distal end portion 3 of the insertion portion 2. An observation window 11, an illuminating window 12, and a fluid supply port 13 that constitute a distal end portion-side connection portion 10 are provided on a distal end side of the protruding portion 3A.

As shown in FIG. 2 and FIG. 3, the observation window 11, the illuminating window 12, and the fluid supply port 13 are arranged at previously determined positions.

More specifically, as shown in FIG. 2, the observation window 11 and the fluid supply port 13 are arranged in first concave portions 3X provided in the distal end face 3B of the protruding portion 3A. The illuminating window 12 is arranged in a second concave portion 3Y provided in a flat portion 3C that is formed in a predetermined shape at a more rear position than the distal end face 3B by notching a portion of the distal end side of the protruding portion 3A.

By adopting this configuration, even if another member contacts the distal end portion 3 of the insertion portion 2 before attaching the optical adapter 4 to the distal end portion 3, another member does not contact the observation window 11, the illuminating window 12, and the fluid supply port 13 that constitute the distal end portion-side connection portion 10. More specifically, the distal end portion-side connection portion 10 can be protected.

Further, as shown in FIG. 3, on a plane including the distal end face 3B and the flat portion 3C of the protruding portion 3A, the observation window 11 and the illuminating window 12, for example, are disposed on a straight line that passes through an axis L of the distal end portion 3. Further, the fluid supply port 13 is arranged at a previously determined position so as to be adjacent to the observation window 11 and the illuminating window 12.

A positioning groove 15 for performing positioning when fixing the optical adapter 4 is provided on a side face portion of the protruding portion 3A of the distal end portion 3. Further, an O-ring 16 is provided on an outer circumferential face of the protruding portion 3A, at a position on a rear end side of the positioning groove 15. The O-ring 16 maintains a space between the distal end portion 3 and the optical adapter 4 in a watertight state when the optical adapter 4 is attached to the distal end portion 3.

An image pickup unit 24 is provided on a rear side (direction towards the interior of the distal end portion) of the observation window 11. The image pickup unit 24 includes an objective optical lens group 25, a solid image pickup device 26, and a control substrate 27 that are located to the rear of the observation window 11. A signal wire that transmits a drive control signal of the solid image pickup device 26 through the control substrate 27, and a signal wire 28 that transmits a video signal of a site to be examined that is generated by the solid image pickup device 26 are connected to the solid image pickup device 26. The signal wire 28 is provided inside a signal cable 29 that is led out from the proximal end side of the image pickup unit 24. The signal cable 29 is inserted through the inside of the insertion portion 2 and guided to the inside of an unshown apparatus main body.

The illuminating window 12 projects an illuminating light onto an observation site via the optical adapter 4. A distal end face of a light guide fiber, which is not shown in the drawings, is disposed at a proximal end face of the illuminating window 12. The light guide fiber is inserted through the inside of the insertion portion 2 and connected to a light source apparatus of the apparatus main body via a connector (unshown).

Further, as shown in FIG. 9, the fluid supply port 13 is a distal end-side opening of a supply passage 30a of an insertion portion-side fluid connection member (hereunder, referred to as “fluid supply section”) 13b. An air/water supply conduit 30 for supplying fluid is arranged in a proximal end-side opening of the supply passage 30a of the fluid supply section 13b. The fluid supply section 13b is a pipe member that has a flange portion at a center in the longitudinal direction, and an O-ring 13a is disposed at an outer circumference thereof that is further on the distal end side than the flange portion.

The air/water supply conduit 30 passes through the inside of the insertion portion 2 and is connected to an unshown air/water supply apparatus inside the apparatus main body. Fluid such as water or air that is supplied from the air/water supply apparatus is delivered to the fluid supply port 13.

When the optical adapter 4 is attached to the distal end portion 3, a space between the fluid supply port 13 and a fluid inlet 20, described later, of the optical adapter 4 that is connected to the fluid supply port 13 is maintained in a watertight state by the O-ring 13a.

In this connection, similarly to the fluid supply port 13, an O-ring may be provided to the observation window 11 and the illuminating window 12, respectively. In addition to ensuring adherence at the time of connection to the optical adapter-side connection portion 17 (see FIG. 4), the O-ring is configured to absorb a contact force at the time of connection, and prevent breakage or the like occurring due to the contact force.

Next, a configuration of the optical adapter 4 is described using FIG. 1 and FIG. 4 to FIG. 9.

Various kinds of optical adapters 4 such as a single lens front-view type, a single lens side-view type, a binocular front-view type, and a binocular side-view type are available as the optical adapter 4. The optical adapter 4 according to the present embodiment is configured as a single lens front-view adapter for observing a site to be examined that is on the front side in the insertion direction.

As shown in FIG. 1 and FIG. 4, the optical adapter 4 has a main body 5 and a retaining ring 6 that has a fitting hole 6A in which the distal end portion 3 of the insertion portion 2 fits. The optical adapter 4 is configured so as to be integrally fixed to the distal end portion 3 by screwing a female screw portion provided on an inner circumferential face of the retaining ring 6 with a male screw portion provided on an outer circumferential face of the distal end portion 3 in a state in which the distal end portion 3 is fitted in the fitting hole 6A.

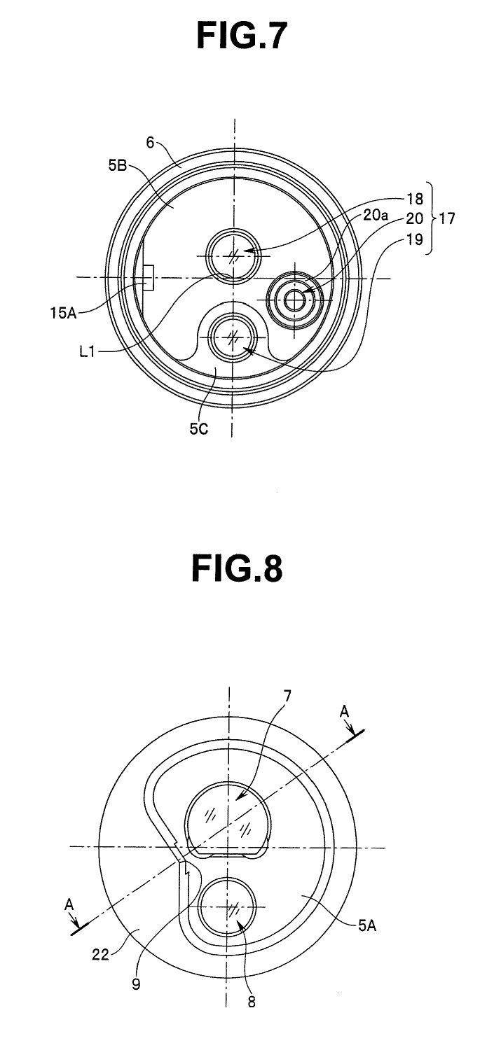

As shown in FIG. 7, a positioning protruding portion 15A is provided on the inner circumferential face of the optical adapter 4. The positioning protruding portion 15A performs positioning when fixing the optical adapter 4 to the distal end portion 3 of the insertion portion 2 by fitting in the positioning groove 15 provided in the distal end portion 3 (see FIG. 2).

As shown in FIG. 9, the main body 5 includes an adapter main body 21 in which the retaining ring 6 is pivotably provided at a proximal end portion, and a distal end cover 22 that is a cover member of the adapter main body 21.

As shown in FIG. 1 and FIG. 6, the distal end cover 22 has an opening 5X that exposes one portion of a distal end face 5A of the adapter main body 21. The distal end cover 22 is fixed to the adapter main body 21 by screwing with a fastening screw 4a that is arranged on a side portion.

As shown by a broken line in FIG. 6, a concave portion 200b that is included in a fluid supply channel 31 is provided at a previously determined position on a back surface side of the distal end cover 22. The concave portion 200b includes a fluid spout 9 for ejecting fluid towards the observation window 7. Further, as shown by a broken line in FIG. 6, an opening 32a of an adapter-side fluid supply conduit 32, which is described later, which is included in the fluid supply channel 31 is provided on the distal end face 5A of the adapter main body 21. The opening 32a communicates with the concave portion 200b (see FIG. 9).

Furthermore, an observation window 7 and an illuminating window 8 that are exposed from the opening 5X are provided in the distal end face 5A of the adapter main body 21.

In this connection, although the fluid spout 9 is formed so as to be capable of ejecting fluid towards the observation window 7, the fluid spout 9 may also be configured to be capable of ejecting fluid not only towards the observation window 7, but also towards the illuminating window 8 at the same time.

According to the present embodiment, as shown in FIG. 1, FIG. 4 and FIG. 9, the optical adapter 4 includes the fluid spout 9 that ejects fluid towards at least the observation window 7, the fluid inlet 20 that connects to and communicates with the fluid supply port 13 for supplying the fluid that is arranged on the distal end face of the distal end portion of the endoscope insertion portion, and the fluid supply channel 31 that allows the fluid inlet 20 and the fluid spout 9 to communicate and through which the fluid flows. The arrangement position of the fluid inlet 20 is common among the plurality of kinds of optical adapters 4, while the fluid spout 9 and the fluid supply channel 31 are configured differently according to the kind of the respective optical adapters 4.

The configuration of the main portions of the optical adapter 4 will now be described using FIG. 4 to FIG. 9.

As shown in FIG. 4 and FIG. 7, an observation window portion 18, an illuminating window portion 19 and the fluid inlet 20 that constitute the optical adapter-side connection portion 17 are provided on a side that is attached to the distal end portion 3 of the insertion portion 2, which is the proximal end side of the optical adapter 4.

The observation window portion 18, the illuminating window portion 19, and the fluid inlet 20 are respectively arranged at positions that conform to the arrangement positions of the observation window 11, the illuminating window 12 and the fluid supply port 13 that constitute the distal end portion-side connection portion 10 of the insertion portion 2.

The observation window portion 18 and the fluid inlet 20 are arranged on a bottom face 5B of the adapter main body 21 that makes surface contacts with the protruding portion 3A of the distal end portion 3. The illuminating window portion 19 is arranged on a protruding portion 5C that protrudes further in the attaching side direction than the bottom face 5B.

Specifically, as shown in FIG. 7, similarly to the connection portion 10 of the distal end portion 3, the observation window portion 18 and the illuminating window portion 19 are, for example, disposed on a straight line that passes through an axis L1 of the optical adapter 4, on a plane including the face of the protruding portion 5C and the bottom face 5B of the adapter main body 21. Further, the fluid inlet 20 is arranged at a previously determined position adjacent to the observation window portion 18 and the illuminating window portion 19.

As shown in FIG. 9, objective lenses 18a and 23 for observing an observation site are arranged in order from the attaching side in the insertion direction in the observation window portion 18. The observation window 7 (see FIG. 1) is provided on the front side in the insertion direction of the objective lens 23.

Further, although not shown in the drawings, a light guide or a lens that transmits an illuminating light is arranged in the illuminating window portion 19. The illuminating window 8 (see FIG. 1) is provided on a front side in the insertion direction of the light guide or the lens.

As shown in FIG. 9, the fluid inlet 20 is a proximal end-side opening of an inlet passage 200a of an adapter-side fluid connection member (hereunder, referred to as fluid inflow portion) 20a. A distal end-side opening of the inlet passage 200a of the fluid inflow portion 20a communicates with the adapter-side fluid supply conduit 32 that is included in the fluid supply channel 31.

The fluid inflow portion 20a is a stepped tubular member, and has a protruding portion 20b that has a thick diameter that protrudes towards the attaching side direction of the distal end portion 3. A concave portion is provided in the protruding portion 20b. The part of the fluid supply section 13b that is on the distal end side than the flange portion is arranged in the concave portion. When the optical adapter 4 is attached to the distal end portion 3, an end face of the protruding portion 20b contacts against the O-ring 13a that is arranged at the fluid supply section 13b, and presses against the O-ring 13a to achieve water-tightness. Thus, fluid that is supplied via the air/water supply conduit 30 is supplied to the adapter-side fluid supply conduit 32 from the fluid supply port 13 via the fluid inlet 20 without leaking to outside.

The adapter-side fluid supply conduit 32 is formed in the adapter main body 21 so as to communicate in a straight line the distal end-side opening of the fluid supply section 20a and the fluid spout 9. That is, according to the present embodiment, the fluid supply channel 31 is constituted by the inlet passage 200a that is formed in the fluid inflow portion 20a, the adapter-side fluid supply conduit 32 that is formed in the adapter main body 21, the concave portion 200b, and the fluid spout 9.

Fluid that is supplied from the fluid supply port 13 to the inlet passage 200a via the fluid inlet 20 passes through the adapter-side fluid supply conduit 32 and the concave portion 200b and is ejected towards the observation window 7 via the fluid spout 9.

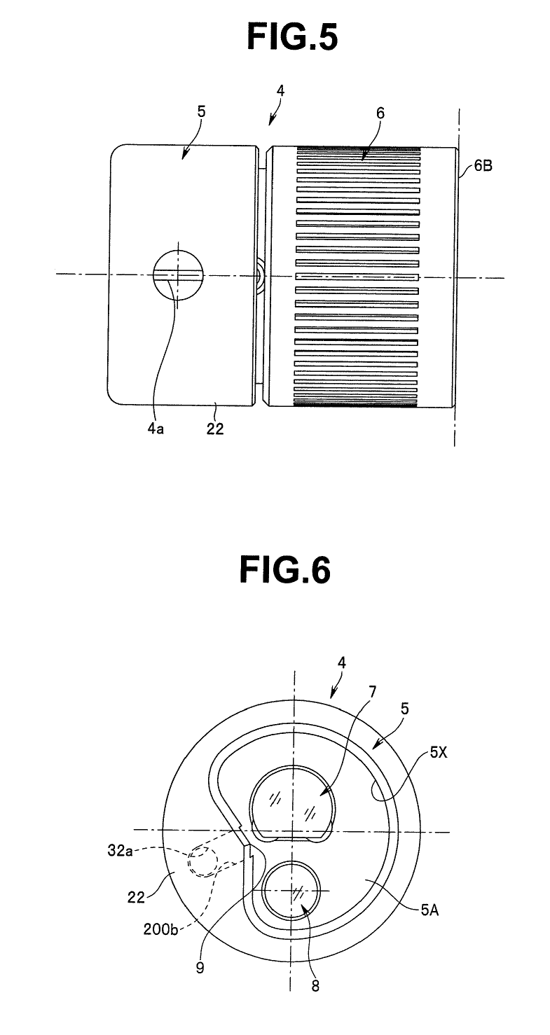

In this connection, similarly to the distal end portion-side connection portion 10, the observation window portion 18, the illuminating window portion 19 and the fluid inlet 20 that constitute the optical adapter-side connection portion 17 are provided at positions that are more recessed than a proximal end face 6B (see FIG. 5) that is formed on an proximal end side of the retaining ring 6 included in the optical adapter 4.

According to this configuration, even if another member contacts the retaining ring 6 of the optical adapter 4 before attaching the optical adapter 4 to the distal end portion 3, the existence of the outer circumferential edge portion that forms the proximal end face 6B of the retaining ring 6 prevents the other member from contacting the observation window portion 18, the illuminating window portion 19 and the fluid inlet 20 that constitute the optical adapter-side connection portion 17. More specifically, the optical adapter-side connection portion 17 can be protected.

Operations of the endoscope apparatus 1 in a state in which the optical adapter 4 having the above configuration is attached to the distal end portion 3 of the insertion portion 2 will now be described using FIG. 1 and FIG. 9.

In the endoscope apparatus 1 shown in FIG. 9, when fluid such as water or air is supplied to the air/water supply conduit 30 from an unshown air/water supply apparatus, the fluid passes through the fluid supply port 13 and the fluid inlet 20 and is fed to the fluid supply channel 31 formed in the optical adapter 4.

The fluid that is fed to the fluid supply channel 31 is supplied to the inlet passage 200a and the adapter-side fluid supply conduit 32, and is ejected towards the observation window 7 from the fluid spout 9 via the concave portion 200b that is formed on a back face of the distal end cover 22 from the opening 32a of the adapter-side fluid supply conduit 32. As a result, dirt adhering to the observation window 7 is removed by the fluid ejected from the fluid spout 9.

Thus, according to the single lens front-view optical adapter 4 of Embodiment 1, since fluid can be appropriately ejected towards the observation window 7 and the like, it is possible to prevent observation from being obstructed by adherents or the like that adhere to the observation window 7, and the observation performance can be improved.

Note that the shape of the fluid spout 9 of the optical adapter 4 is not limited to the shape shown in FIG. 1 and FIG. 6, and the fluid ejecting strength or the ejecting direction can be set as desired by suitably changing the shape thereof.

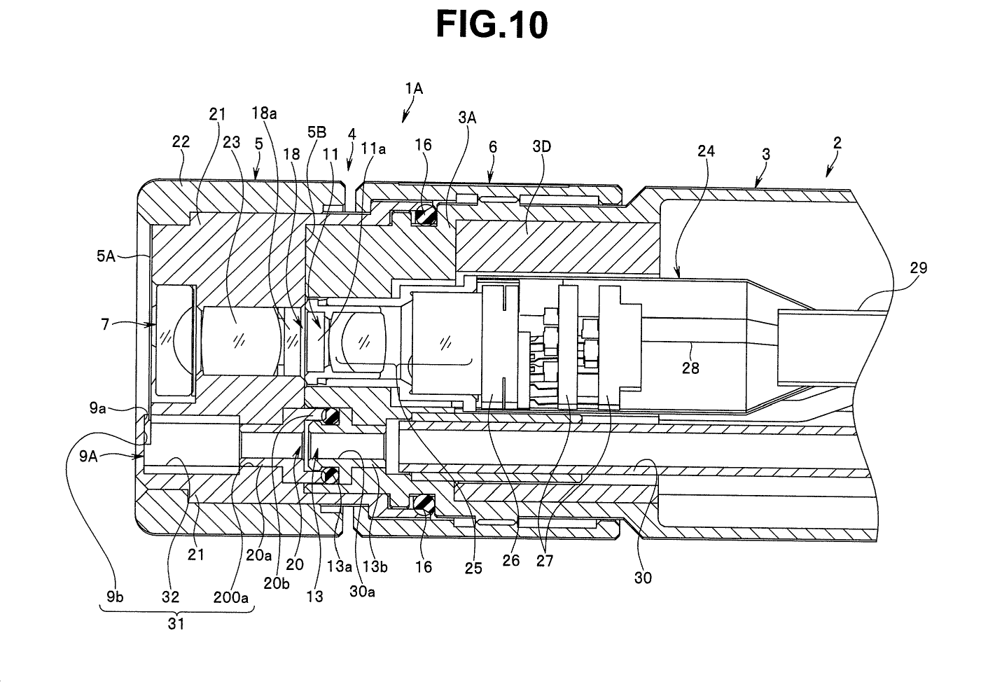

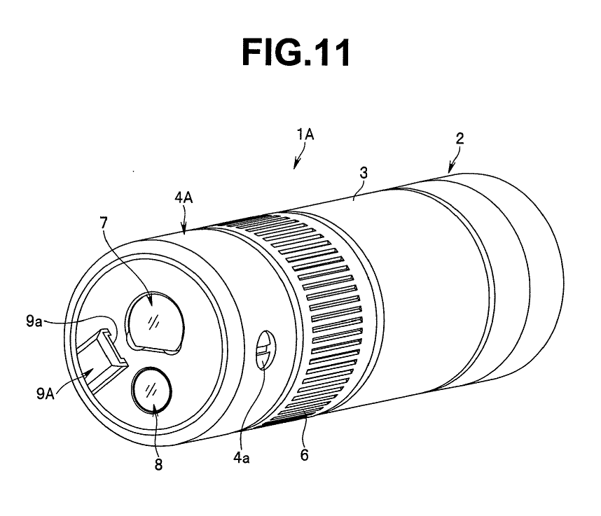

For example, the fluid spout 9 can be configured as shown in FIG. 10 and FIG. 11. In the optical adapter 4 shown in FIG. 10, the fluid spout 9 is formed integrally with the adapter main body 21. As a result, the same actions and advantages as described above can be obtained.

According to an optical adapter 4A of an endoscope apparatus 1A shown in FIG. 11, a fluid spout member 9A is provided in a fixed condition by means of an adhesive or a welding at a predetermined position on the distal end face 5A of an adapter main body 22. The fluid spout member 9A has a fluid passage and a fluid spout 9a that communicate with the opening 32a of the adapter-side fluid supply conduit 32. According to this configuration, although the number of components increases, the same actions and advantages as in the above described Embodiment 1 can be easily obtained.

In this connection, according to the present embodiment, the arrangement of the observation window 11, the illuminating window 12, and the fluid supply port 13 in the distal end portion 3 of the insertion portion 2 is not limited to the positions shown in FIG. 3. For example, the arrangement positions of the observation window 11, the illuminating window 12, and the fluid supply port 13 may be suitably changed based on a configuration that is suited to reducing the diameter of the insertion portion 2. Likewise, the arrangement positions of the observation window portion 18, the illuminating window portion 19, and the fluid inlet 20 in the optical adapter 4 are also not limited to the positions shown in FIG. 7. In this connection, when changing the positions, a configuration is adopted such that the arrangement positions of the observation window 11, the illuminating window 12, and the fluid supply port 13 that constitute the distal end portion-side connection portion 10 of the insertion portion 2 and the arrangement positions of the observation window portion 18, the illuminating window portion 19, and the fluid inlet 20 in the optical adapter 4 are in conformity with each other.

### Embodiment 2

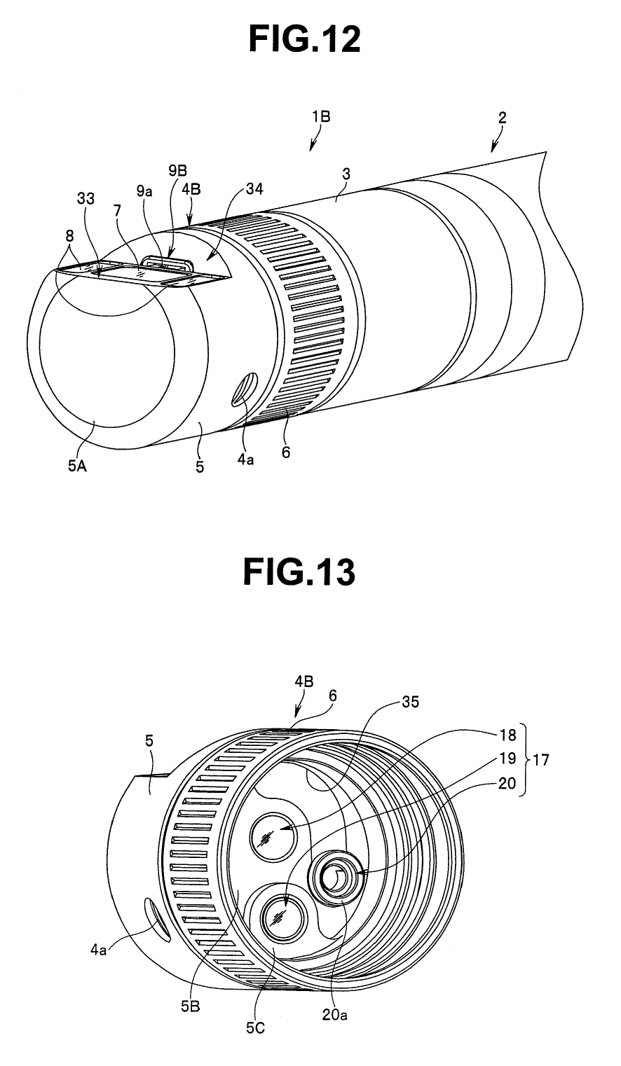

FIG. 12 to FIG. 18 illustrate Embodiment 2 of the endoscope apparatus according to the present invention. In this connection, in the following description of an endoscope apparatus 1B illustrated in FIG. 12 to FIG. 18, components that are the same as in the foregoing Embodiment 1 are denoted by the same reference symbols and a description of those components is omitted, and only portions that are different to that of Embodiment 1 are described.

The endoscope apparatus 1B of the present embodiment has the same insertion portion 2 as those of Embodiment 1. Instead of the single lens front-view type optical adapter 4, an optical adapter 4B that is a single lens side-view adapter that performs observation of a site to be examined in a direction orthogonal to the insertion direction is provided on the insertion portion 2.

As shown in FIG. 12 to FIG. 18, a side-view face 33 and an inclined face 34 are provided in the main body 5 of the optical adapter 4B. The side-view face 33 and the inclined face 34 are formed by notching a part of a side face portion of the main body 5. The side-view face 33 is oriented in a direction orthogonal to the insertion direction. The inclined face 34 is formed in a diagonal direction with respect to the side-view face 33.

The observation window 7 is provided at substantially the center of the side-view face 33. Illuminating windows 8 are respectively provided on the two sides of the observation window 7 of the side-view face 33.

A fluid spout member 9B that has the fluid spout 9a is provided on the side-view face 33 side of the inclined face 34. A flow channel 201b that communicates with the fluid spout 9a is formed in the fluid spout member 9B. Another end of the flow channel 201b communicates with an adapter-side fluid supply conduit 32A, described later, which constitutes a fluid supply channel 31A.

Therefore, the configuration is such that when fluid is supplied through the fluid supply channel 31A, the fluid is ejected towards the observation window 7 from the fluid spout 9a of the fluid spout member 9B. In this connection, similarly to Embodiment 1, an ejecting direction of the fluid spout 9a is not limited to the direction of the observation window 7, and a configuration may be adopted in which fluid is also ejected towards the illuminating windows 8 to thereby enable the ejection of fluid towards the windows 7 and 8.

According to the present embodiment, as shown in FIG. 13 to FIG. 16, the observation window portion 18, the illuminating window portion 19 and the fluid inlet 20 that constitute the optical adapter-side connection portion 17 are provided on the proximal end side of the optical adapter 4B in substantially the same condition as in Embodiment 1.

The fluid supply channel 31A of the optical adapter 4B is used for a single lens side-view type optical adapter, and is formed so that fluid can be appropriately ejected towards the observation window 7 and the like.

The fluid supply channel 31A of the optical adapter 4B includes the flow channel 201b of the fluid spout member 9B, a groove channel 35A, an inlet passage 201a of a fluid inflow portion 20a, and the adapter-side fluid supply conduit 32A. The fluid inflow portion 20a is constituted by the inlet passage 201a and the adapter-side fluid supply conduit 32A.

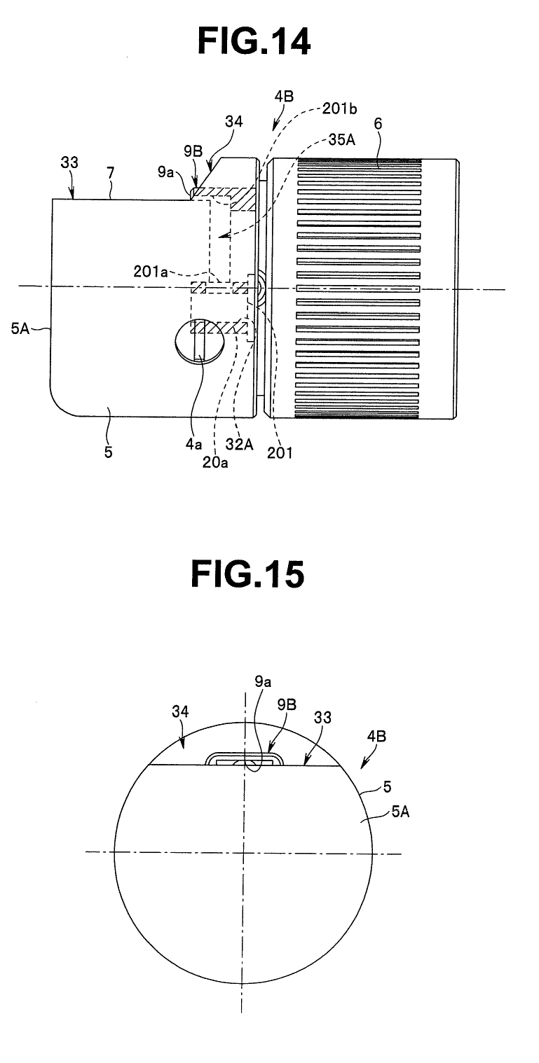

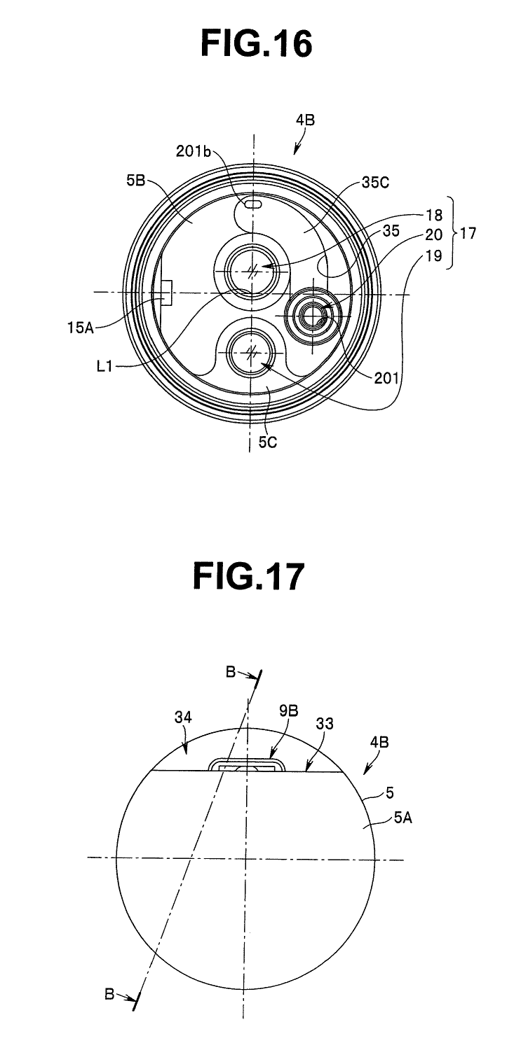

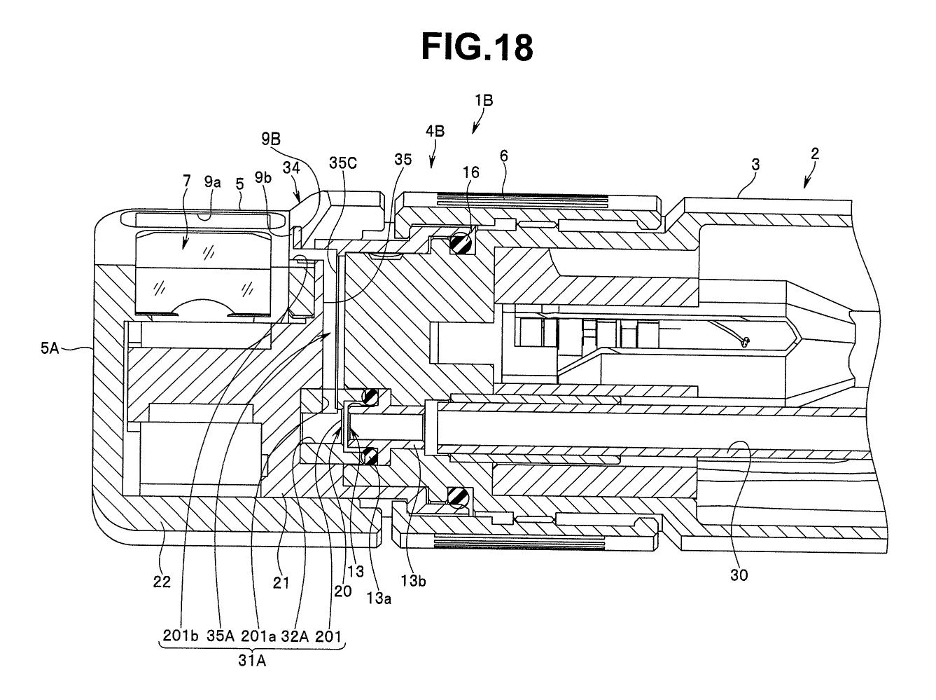

More specifically, as shown in FIG. 13, FIG. 14, FIG. 16 and FIG. 18, a groove channel portion 35 that is one part of the fluid supply channel 31 is provided on a bottom face 5B that is an end face that has the fluid inlet 20 of the optical adapter 4B. That is, the groove channel portion 35 constituting the groove channel 35A that is one portion of the fluid supply channel 31 is formed on the bottom face 5B of the optical adapter 4B.

As shown in FIG. 18, the groove channel portion 35 is formed in a groove shaped by recessing one portion of the bottom face 5B of the adapter main body 21, and communicates with the inlet passage 201a of the fluid inflow portion 20a and the adapter-side fluid supply conduit 32A. As shown in FIG. 16, on a flat surface of the bottom face 5B, the groove channel portion 35 is formed in a curved shape so as to cause the fluid inlet 20 and the flow channel 201b to communicate.

Further, the groove channel portion 35 constitutes the groove channel 35A that is one portion of the fluid supply channel 31 by adhering thereto a cover 35C that is aligned with a groove of the groove channel portion 35.

A curved shape of the groove channel portion 35 is not limited to the shape shown in FIG. 13 and FIG. 16, and a configuration may be adopted that is appropriately changed in accordance with arrangement positions of the observation window portion 18, the illuminating window portion 19, and the fluid inlet 20 that are suited to reducing the diameter of the optical adapter 4 or the like.

Further, to prevent leakage of fluid from the fluid supply channel 31 by means of the groove channel portion 35, a configuration may be adopted in which, instead of the cover 35C, for example, an O-ring is provided at an outer circumferential edge portion of the groove channel portion 35 to make a space between the distal end face 3B of the distal end portion 3 and the bottom face 5B of the optical adapter 4B watertight, to thereby seal the groove channel 35A with greater precision by means of the groove channel portion 35.

The remaining configuration is the same as Embodiment 1.

Next, operations of the endoscope apparatus 1B according to Embodiment 2 will be described. In the endoscope apparatus 1B shown in FIG. 18, fluid such as water or air is supplied to an air/water supply conduit 30 from an unshown air/water supply apparatus. The fluid passes through a fluid supply port 13 and the fluid inlet 20 and is fed to the fluid supply channel 31A.

The fluid that is fed to the fluid supply channel 31A is supplied to the adapter-side supply conduit 32a, the inlet passage 201a, the groove channel 35A formed by the groove channel portion 35, and the flow channel 201b formed in the fluid spout member 9B, and is sent to the fluid spout 9a of the fluid spout member 9B and ejected towards the observation window 7. As a result, dirt adhering to the observation window 7 is removed by the ejected fluid.

Thus, according to Embodiment 2, even when the optical adapter 4 is a single lens side-view type optical adapter, by providing the fluid supply channel 31A in the optical adapter it is possible to appropriately eject fluid towards the observation window 7 and the like, and hence the observation performance can be improved.

Note that according to the present embodiment also, the shape of the fluid spout 9a of the optical adapter 4B is not limited to the shape shown in FIG. 12 and FIG. 15, and a configuration may be adopted in which the fluid ejecting strength or the ejecting direction is set as desired by suitably changing the shape thereof.

### Embodiment 3

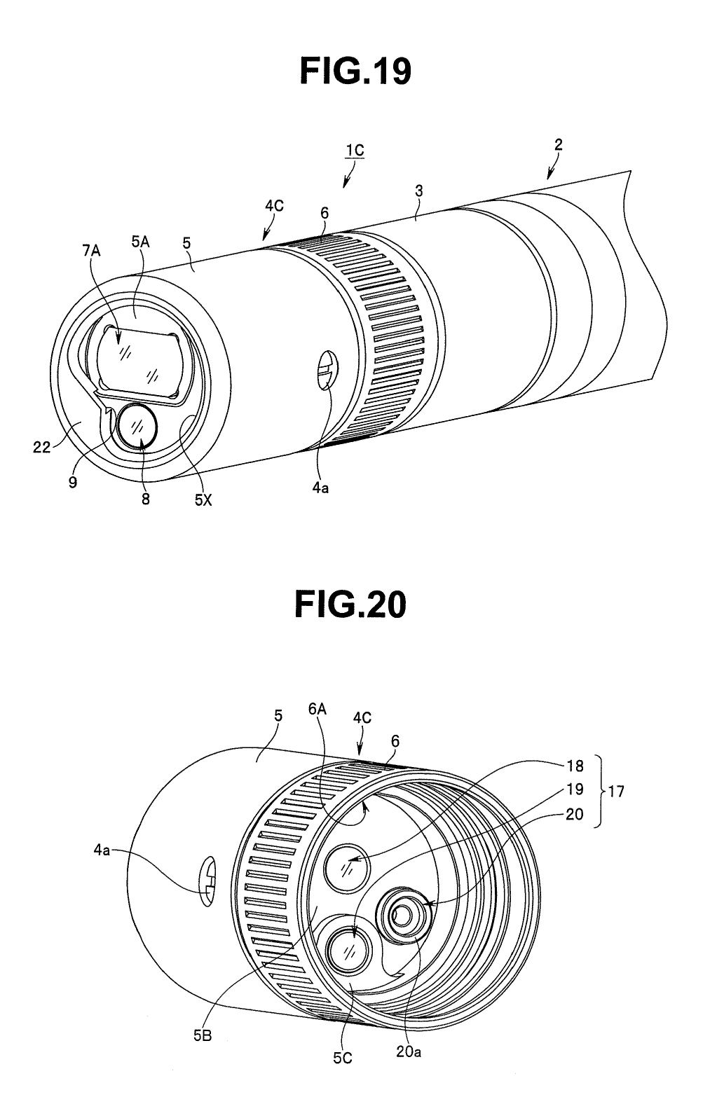

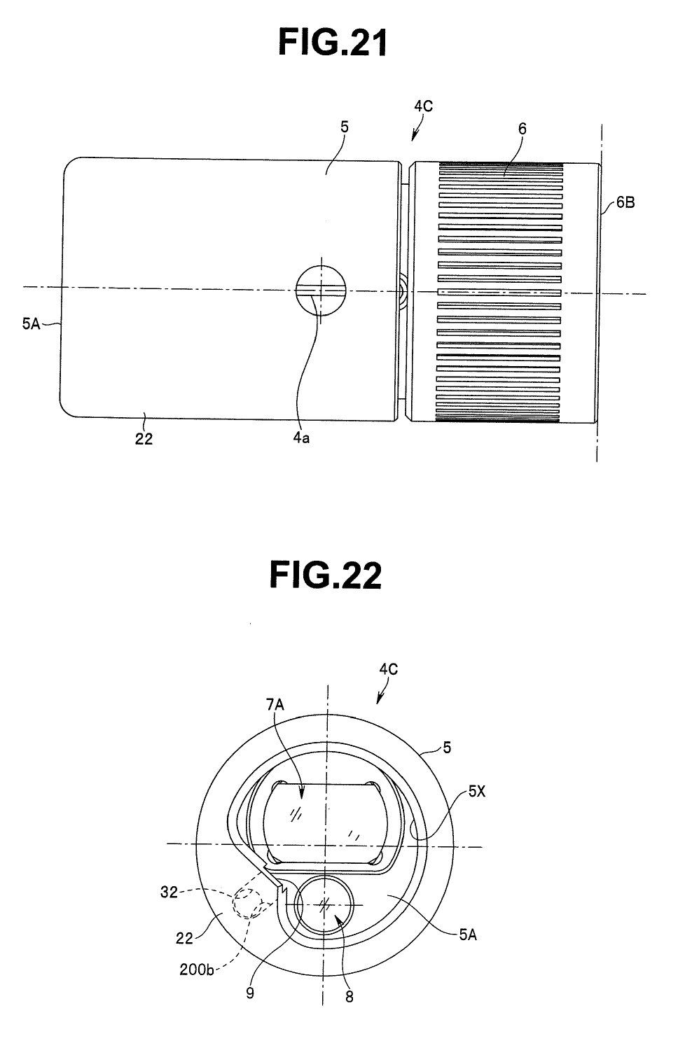

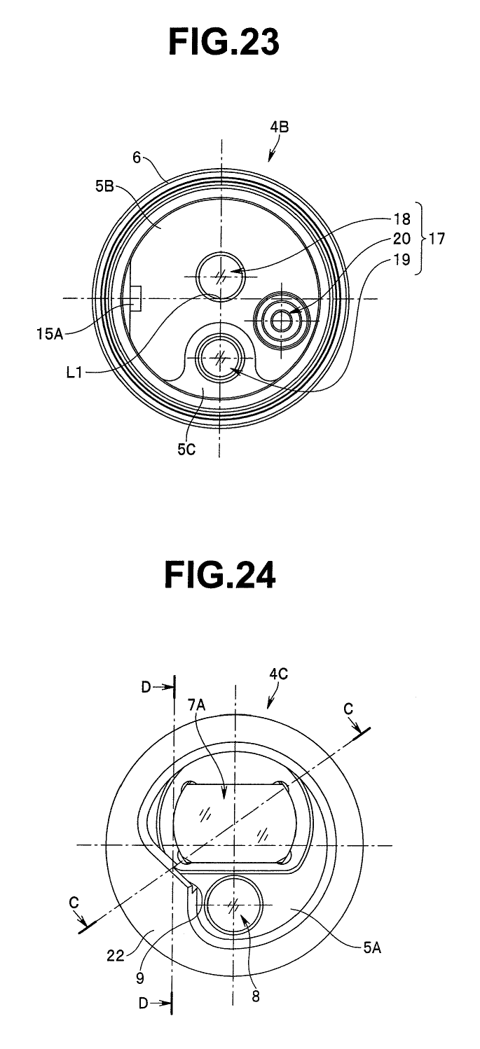

Embodiment 3 of the endoscope apparatus according to the present invention is illustrated in FIG. 19 to FIG. 26. In this connection, in the following description of an endoscope apparatus 1C illustrated in FIG. 19 to FIG. 26, components that are the same as in the foregoing embodiments are denoted by the same reference symbols and a description of those components is omitted, and only portions that are different to those of the foregoing embodiments are described.

The endoscope apparatus 1C of the present embodiment has the same insertion portion 2 as those of Embodiments 1 and 2. Instead of the above described optical adapter 4, an optical adapter 4C that is a binocular front-view adapter for performing binocular observation of a site to be examined that is at a forward position in the insertion direction is provided on the insertion portion 2.

As shown in FIG. 19 and FIG. 22, although the configuration of the main body 5 of the optical adapter 4C is substantially the same as in Embodiment 1 above, instead of the observation window 7, a binocular observation window 7A is provided on the distal end face 5A of the adapter main body 21.

Further, with respect to the fluid spout 9 formed by the distal end cover 22 also, although the configuration is substantially the same as that of the above Embodiment 1, the shape of the fluid spout 9 is formed in conformity with the binocular observation window 7A whose area is greater than that of the observation window 7.

More specifically, even in the case of the binocular observation window 7A that has a large area, the fluid spout 9 is configured so that fluid can be reliably ejected towards the binocular observation window 7A.

According to the present embodiment, since the optical adapter 4C is a binocular front-view adapter, an objective lens and a binocular optical unit 40 that is required for performing binocular observation of a site to be examined that is forward of the optical adapter 4C in the insertion direction are arranged in the binocular observation window 7A.

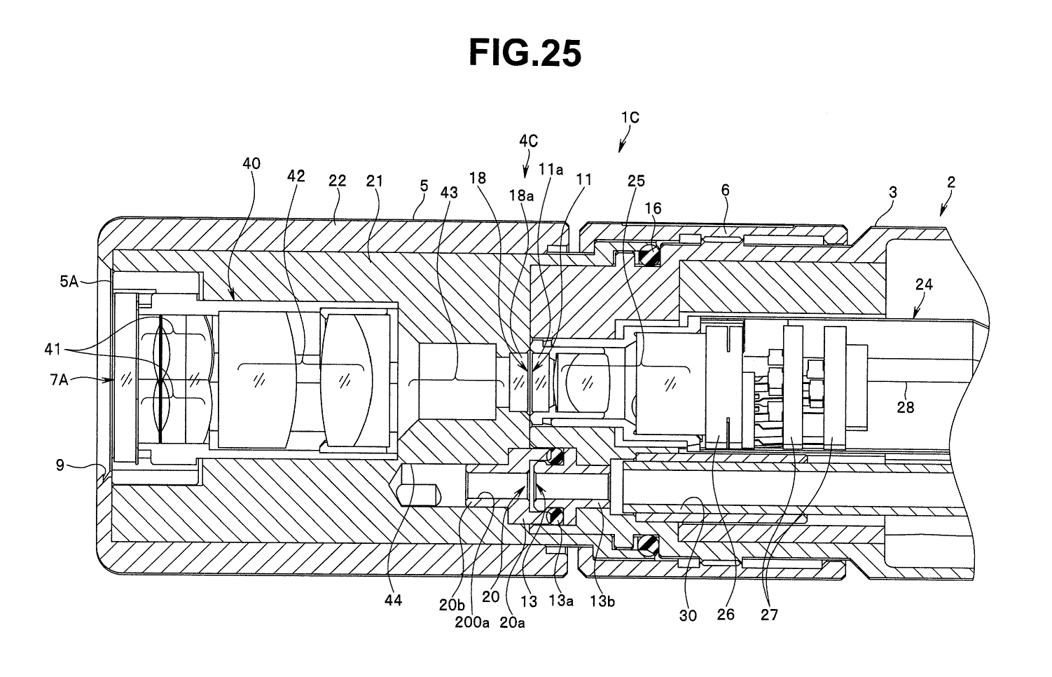

As shown in FIG. 25, the binocular optical unit 40 is arranged to a rear of the binocular observation window 7A, and includes a pair of left and right objective optical lenses 41 that are required in order to capture video of a site to be examined as video light for binocular viewing, and a first objective lens group 42 and a second objective lens group 43 that are arranged to a rear of the objective optical lenses 41. The observation window portion 18 is arranged to a rear of the second objective lens group 43 via an objective lens 18a.

According to the present embodiment, similarly to Embodiment 1, the observation window portion 18, the illuminating window portion 19, and the fluid inlet 20 that constitute the optical adapter-side connection portion 17 are provided on the proximal end side of the optical adapter 4C.

In this case, as described above, since the binocular optical unit 40 for a binocular front-view is provided inside the main body of the binocular front-view optical adapter 4C, the length of the main body 5 in the insertion axis direction (longitudinal direction) is increased, and a space to arrange the adapter-side fluid supply conduit 32 for supplying fluid is limited.

Although the optical adapter 4C of the present embodiment is a binocular front-view optical adapter, the optical adapter 4C includes a fluid supply channel 31B that can appropriately eject fluid towards the binocular observation window 7A and the like.

The fluid supply channel 31B of the optical adapter 4C is formed to include a conduit in which a channel that links the fluid inlet 20 and the fluid spout 9 is curved so as to correspond to a structure of the binocular front-view optical adapter 4C.

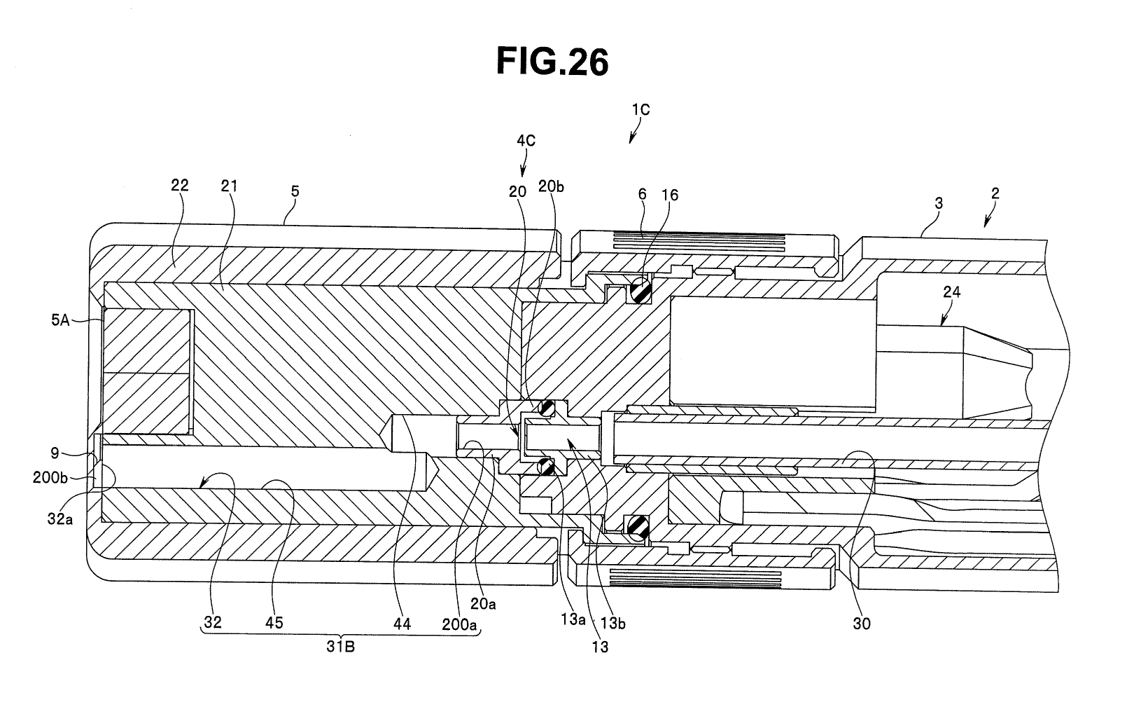

More specifically, as shown in FIG. 25 and FIG. 26, the fluid supply channel 31B includes the inlet passage 200a of the fluid inflow portion 20a, a first supply conduit 44 that is linearly provided along the insertion direction that communicates with the fluid inlet 20, a second supply conduit 45 that is linearly provided along an opposite direction to the insertion direction from the distal end face 5A via the fluid spout 9, the concave portion 200b, and the fluid spout 9.

As shown in FIG. 26, a central axis of the first supply conduit 44 and a central axis of the second supply conduit 45 are out of alignment with each other in a direction orthogonal to the insertion direction. The first supply conduit 44 and the second supply conduit 45 are caused to communicate by being formed so that a portion of the first supply conduit 44 and a portion of the second supply conduit 45 overlap.

The first supply conduit 44 included in the fluid supply channel 31B is formed by performing a boring operation from the bottom face 5B side that is the proximal end face of the adapter main body 21. In contrast, the second supply conduit 45 is formed by performing a boring operation from the distal end face 5A side of the adapter main body 21 so that one portion of the second supply conduit 45 overlaps with the first supply conduit 44. Accordingly, there is the advantage that the fluid supply channel 31B can be formed more simply than the curved fluid supply channel 31A of Embodiment 2.

The remaining configuration is the same as that of Embodiment 1.

Next, the operations of the endoscope apparatus 1C of Embodiment 3 are described.

In the endoscope apparatus 1C shown in FIG. 26, when fluid such as water or air is supplied to the air/water supply conduit 30 from an unshown air/water supply apparatus, the fluid passes through the fluid supply port 13 and the fluid inlet 20 and is fed to the fluid supply channel 31B formed in the optical adapter 4.

The fluid that is fed to the fluid supply channel 31B passes through the inlet passage 200a, the first supply conduit 44, the second supply conduit 45, and the opening 32a of the adapter fluid supply conduit 32, and is ejected towards the binocular observation window 7A from the fluid spout 9 via the concave portion 200b formed in the back face of the distal end cover 22. As a result, dirt adhering to the binocular observation window 7A is removed by the fluid ejected from the fluid spout 9.

Thus, according to Embodiment 3, even when the optical adapter 4 is a binocular front-view optical adapter, by providing the fluid supply channel 31B in the optical adapter, it is possible to appropriately eject fluid towards the binocular observation window 7A and the like, and hence the observation performance can be improved.

Note that according to the present embodiment also, the shape of the fluid spout 9 of the optical adapter 4C is not limited to the shape shown in FIG. 19 and FIG. 22, and a configuration may be adopted in which the fluid ejecting strength or the ejecting direction is set as desired by suitably changing the shape thereof.

Further, in addition to the shape of the fluid spout, with respect to the first supply conduit 44 and the second supply conduit 45 of the adapter-side fluid supply conduit 32 also, a configuration may be adopted in which the diameter of the supply conduits is suitably changed so as to obtain a required fluid supply pressure or the like.

### Embodiment 4

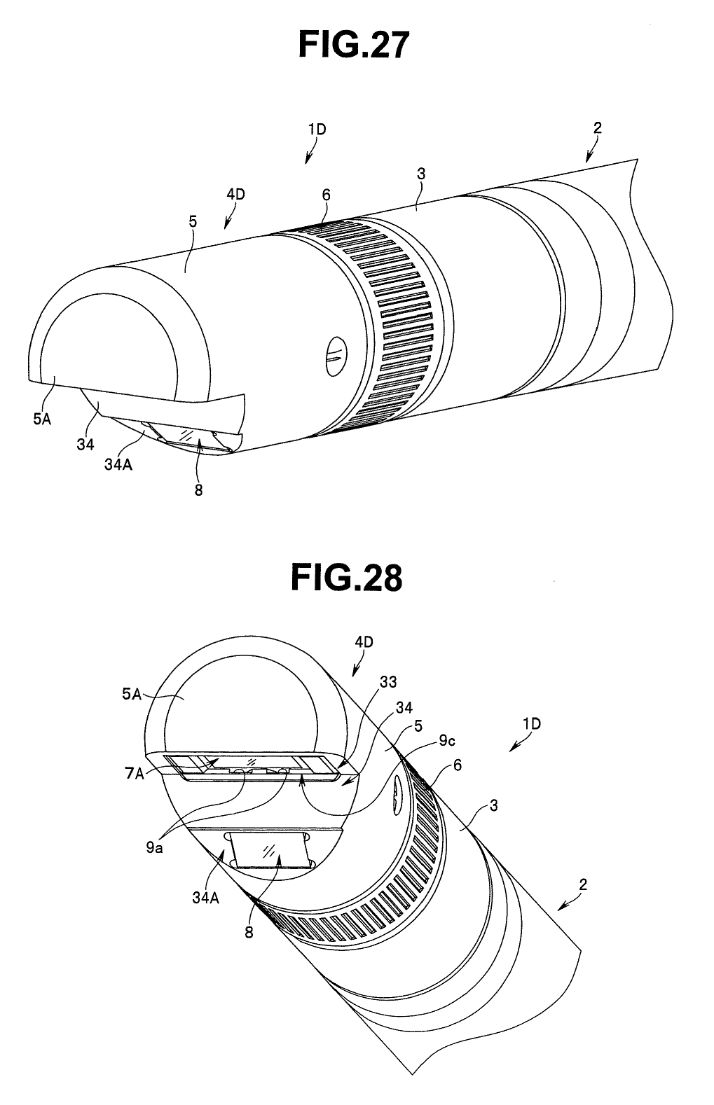

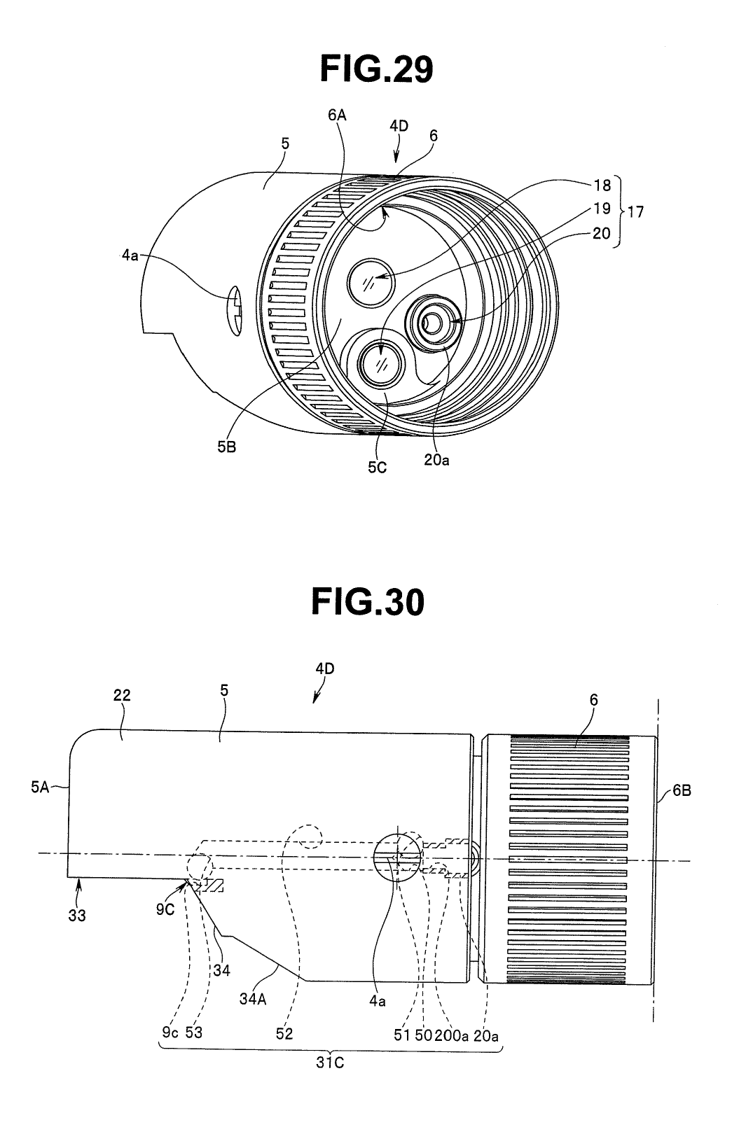

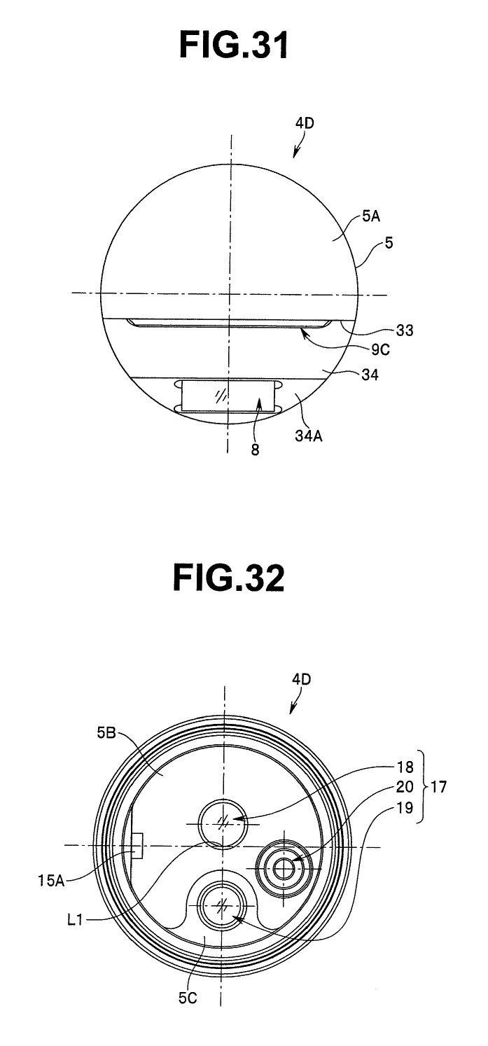

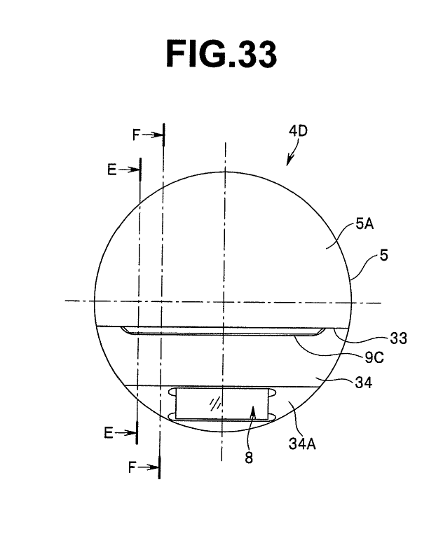

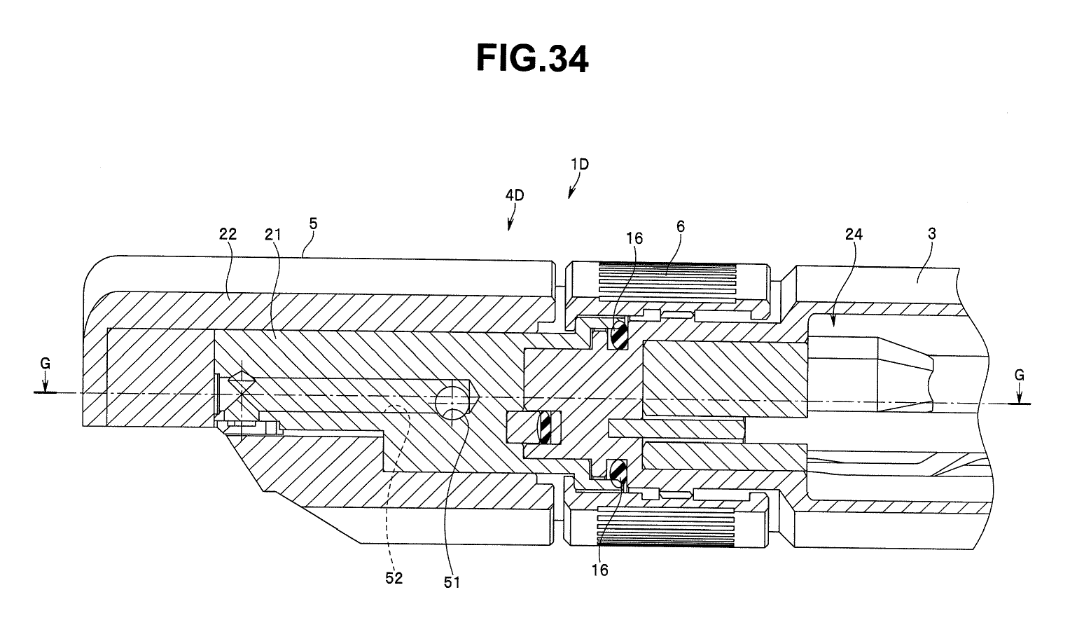

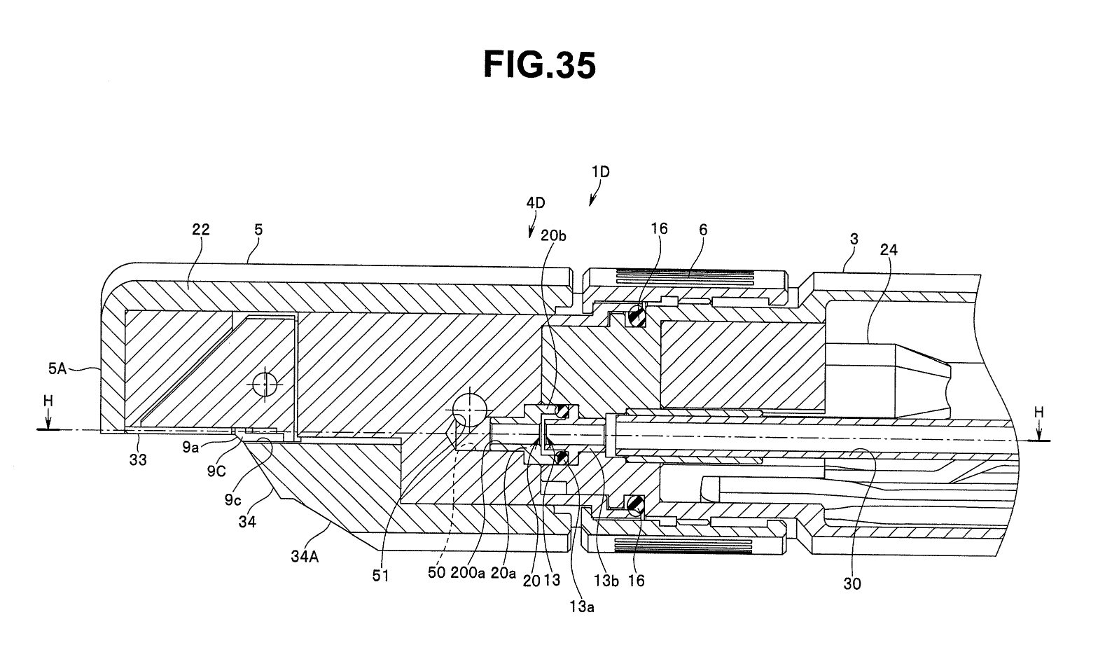

FIG. 27 to FIG. 37 illustrate Embodiment 4 of the endoscope apparatus according to the present invention. In this connection, in the following description of an endoscope apparatus 1D illustrated in FIG. 27 to FIG. 37, components that are the same as in the foregoing embodiments are denoted by the same reference symbols and a description of those components is omitted, and only portions that are different to those of the foregoing embodiments are described.

The endoscope apparatus 1D of the present embodiment has the same insertion portion 2 as those of Embodiments 1 and 2. Instead of the above described single lens side-view optical adapter 4B, the insertion portion 2 is provided with an optical adapter 4D that is a binocular side-view adapter for performing binocular observation of a site to be examined in a direction orthogonal to the insertion direction.

As shown in FIG. 27 to FIG. 28, although the configuration of the main body 5 of the optical adapter 4D is substantially the same as that of the foregoing Embodiment 2, in addition to the side-view face 33 and the inclined face 34, a second inclined face 34A that links to the inclined face 34 is newly provided in the optical adapter 4D. The second inclined face 34A is formed in a direction that inclines further from the inclined face 34.

Further, the binocular observation window 7A is provided instead of the observation window 7 (see FIG. 12) on the side-view face 33. In addition, the two illuminating windows 8 (see FIG. 12) provided on the side-view face 33 are eliminated, and one illuminating window 8 is newly provided on the second inclined face 34A.

More specifically, by adopting a configuration in which the illuminating window 8 is arranged at a position that is further to a rear in the opposite direction to the insertion direction than the binocular observation window 7A of the optical adapter 4D, a site to be examined of a wider range can be illuminated and binocular observation can be performed with precision.

With respect to fluid spouts 9a of a fluid spout member 9C provided on the side-view face 33 side of the inclined face 34 also, although the configuration thereof is substantially the same as the fluid spout 9a of the foregoing Embodiment 2, the fluid spouts 9a are formed so as to conform to the binocular observation window 7A whose area is greater than that of the observation window 7.

More specifically, the fluid spout member 9C is provided with two fluid spouts 9a so that, even in the case of the binocular observation window 7A that has a large area, fluid can be reliably ejected towards the binocular observation window 7A.

According to the present embodiment, since the optical adapter 4D is a binocular side-view optical adapter, an objective lens (not shown) and a binocular optical unit 40A that is required for performing binocular observation of a site to be examined in a direction orthogonal to the insertion direction are arranged in the binocular observation window 7A.

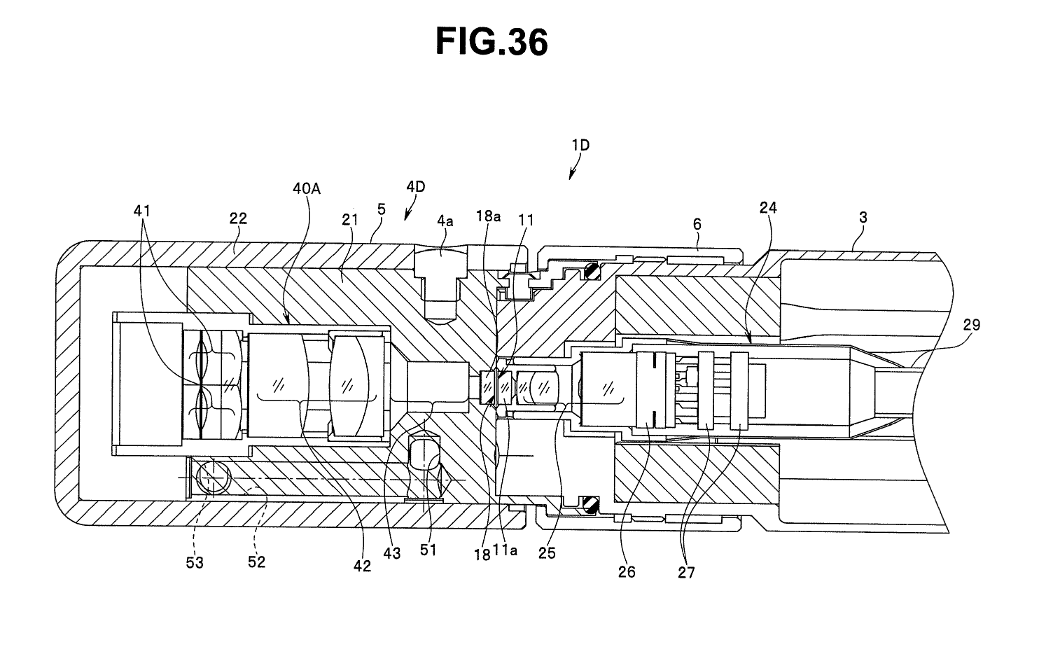

As shown in FIG. 36, the binocular optical unit 40A is arranged to the rear of the binocular observation window 7A, and includes a pair of left and right objective optical lenses 41 that are required in order to capture video of a site to be examined as video light for binocular viewing, and a first objective lens group 42 and a second objective lens group 43 that are arranged to the rear of the objective optical lenses 41. The observation window portion 18 is arranged to the rear of the second objective lens group 43 via the objective lens 18a.

According to the present embodiment, similarly to Embodiment 2, the observation window portion 18, the illuminating window portion 19, and the fluid inlet 20 that constitute the optical adapter-side connection portion 17 are provided on the proximal end side of the optical adapter 4D.

As described above, since the binocular optical unit 40A for a binocular side-view is provided inside the main body of the binocular side-view optical adapter 4D, the length of the main body 5 in the insertion axis direction (longitudinal direction) is increased, and a space to arrange the adapter-side fluid supply conduit 32 for supplying fluid is limited.

Although the optical adapter 4D of the present embodiment is a binocular side-view optical adapter, the optical adapter 4D includes a fluid supply channel 31C that can appropriately eject fluid towards the binocular observation window 7A and the like.

The fluid supply channel 31C of the optical adapter 4D is formed to include a conduit in which a channel that links the fluid inlet 20 and the fluid spout 9a is curved so as to correspond to the structure of the binocular side-view optical adapter 4D.

More specifically, as shown in FIG. 30 and FIG. 34 to FIG. 37, the fluid supply channel 31C includes the inlet passage 200a of the fluid inflow portion 20a, a first supply conduit 50 that is linearly provided along the insertion direction through the fluid inlet 20, the concave portion 200b, the fluid spouts 9a, the conduit 9c provided in the fluid spout member 9C, a second supply conduit 52 that is linearly provided along an opposite direction to the insertion direction and that communicates with the fluid spouts 9a, a third supply conduit 51 that is provided in a direction orthogonal to the insertion direction and that causes the first supply conduit 50 and the second supply conduit 52 to communicate, and a fourth supply conduit 53 that is provided in a direction orthogonal to the insertion portion and that causes the conduit 9c and the second supply conduit 52 to communicate.

The first supply conduit 50 included in the fluid supply channel 31C is formed by performing a boring operation from the bottom face 5B side that is the proximal end face of the adapter main body 21. In contrast, the second supply conduit 52 is formed by performing a boring operation from the distal end face 5A side of the adapter main body 21.

Further, the third supply conduit 51 is formed by performing a boring operation so as to cause the first supply conduit 50 and the second supply conduit 52 to communicate, from a side face on the proximal end side of the adapter main body 21. In contrast, the fourth supply conduit 53 is formed by performing a boring operation so as to cause the conduit 9c, which supplies fluid to the fluid spout 9a, and the second supply conduit 51 to communicate, from a side face on the distal end side of the adapter main body 21.

In this connection, after completing the respective operations to bore the third supply conduit 51 and the fourth supply conduit 53, hole plugging operations are performed in which the respective openings provided in the side face portions of the adapter main body 21 are sealed using a lid member such as a resin or a filling member such as an adhesive.

The remaining configuration is the same as that of Embodiment 2.

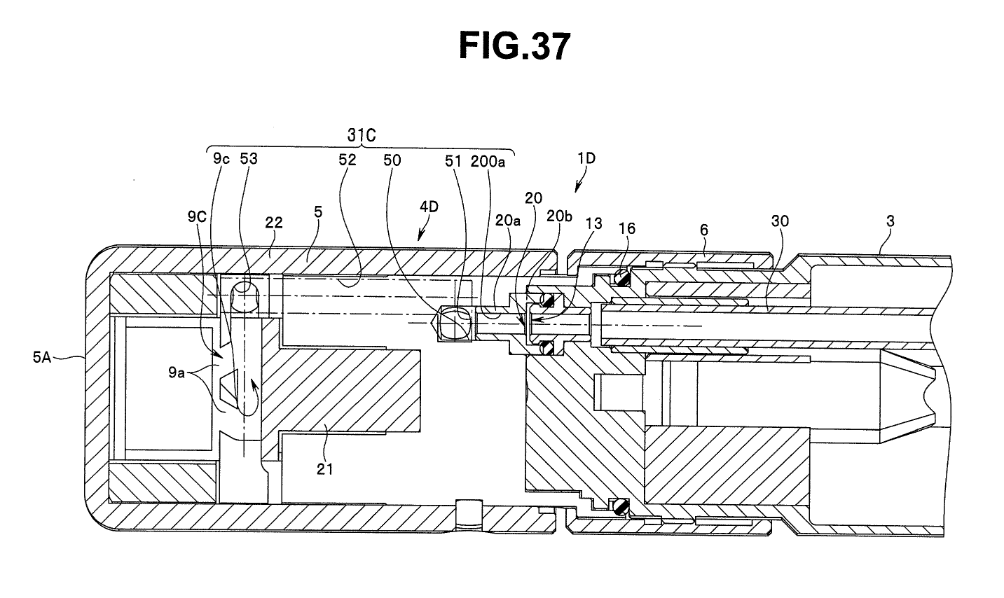

Next, the operations of the endoscope apparatus 1D of Embodiment 4 are described. In the endoscope apparatus 1D shown in FIG. 37, when fluid such as water or air is supplied to the air/water supply conduit 30 from an unshown air/water supply apparatus, the fluid passes through the fluid supply port 13 and the fluid inlet 20 and is fed to the fluid supply channel 31C.

As shown in FIG. 30 and FIG. 34 to FIG. 37, the fluid that is fed to the fluid supply channel 31C passes through the inlet passage 200a, the first supply conduit 50, the second supply conduit 52, the third supply conduit 51, the fourth supply conduit 53, and the conduit 9c of the fluid spout member 9C, and is ejected towards the binocular observation window 7A from the two fluid spouts 9a. As a result, dirt adhering to the binocular observation window 7A is removed by the fluid ejected from the fluid spouts 9a.

Thus, according to Embodiment 4, even when the optical adapter 4 is the binocular side-view optical adapter 4D, by providing the fluid supply channel 31C in the optical adapter, it is possible to appropriately eject fluid towards the binocular observation window 7A and the like, and hence the observation performance can be improved.

Note that according to the present embodiment also, the shape of the fluid spouts 9a of the optical adapter 4D is not limited to the shape shown in FIG. 28 and FIG. 37, and a configuration may be adopted in which the fluid ejecting strength or the ejecting direction is set as desired by suitably changing the shape thereof.

Further, in addition to the shape of the fluid spouts 9a, with respect to the first supply conduit 50 to the fourth supply conduit 53 of the fluid supply channel 31C also, a configuration may be adopted in which the diameter of the supply conduits is suitably changed or the length is changed so as to obtain a required fluid supply pressure or the like.

Furthermore, although according to the present embodiment the two fluid spouts 9a are configured so as to eject fluid towards only the binocular observation window 7A, a configuration may also be adopted that enables a fluid to be ejected towards the illuminating window 8 at the same time by, for example, providing a dedicated fluid supply conduit for the illuminating window 8 that branches from the fluid supply channel 31C as well as a dedicated fluid spout for the illuminating window 8 that communicates with the fluid supply conduit on the second inclined face 34.

Note that, according to the present invention, when a configuration is adopted in which the fluid supply channel 31 bends or curves, the shape of the fluid supply channel 31 is not limited to a shape shown in Embodiments 2 to 4, and even when another shape is adopted, for example, even when the optical adapter 4 has a reduced diameter, the fluid supply channel 31 may be configured using a flexible, elastic conduit or the like that can be freely arranged so as to run along a space inside the optical adapter 4.

The present invention is not limited to the above described embodiments and modification examples, and various changes and modifications are possible within a range that does not depart from the spirit and the scope of the present invention.

What is claimed is:

1. An endoscope apparatus comprising a plurality of kinds of optical adapters in which an illuminating window that radiates an illuminating light and an observation window through which a subject can be viewed are provided at previously determined positions, and an endoscope insertion portion having a distal end portion to which the plurality of optical adapters can be detachably attached, wherein:
in a configuration in which a fluid spout that ejects fluid at least at a position corresponding to the observation window is provided in each of the optical adapters,
the endoscope insertion portion comprises a fluid supply port for supplying the fluid to a previously determined position on a distal end face of the distal end portion; and
the plurality of kinds of optical adapters comprise:
a fluid inlet that is connected to the fluid supply port, and
a fluid supply channel that causes the fluid inlet and the fluid spout that is provided in each of the optical adapters to communicate, and that differs according to a kind of the optical adapter.

2. The endoscope apparatus according to claim 1, wherein:
an optical adapter-side connection portion for connecting the optical adapter to the distal end portion of the endoscope insertion portion, which includes the fluid inlet, is provided at a position that is more recessed than a proximal end face on a attaching side of the optical adapter; and
a distal end portion-side connection portion for connecting the distal end portion to the optical adapter, which includes the fluid supply port, is provided at a position that is more recessed than a proximal end face on a attaching side of the distal end portion.

3. The endoscope apparatus according to claim 1, wherein:
the fluid supply channel comprises:
an inlet passage that is formed in the fluid inlet and that is formed so as to cause the fluid inlet and the fluid spout to communicate in a straight line, an adapter-side supply conduit that is formed in the optical adapter main body, a concave portion provided in a distal end portion of the optical adapter, and the fluid spout;
wherein the fluid spout is configured so as to also eject the fluid towards the illuminating window.

4. The endoscope apparatus according to claim 2, wherein:
the fluid supply channel comprises:
an inlet passage that is formed in the fluid inlet and that is formed so as to cause the fluid inlet and the fluid spout to communicate in a straight line, an adapter-side supply conduit that is formed in the optical adapter main body, a concave portion provided in a distal end portion of the optical adapter, and the fluid spout;
wherein the fluid spout is configured so as to also eject the fluid towards the illuminating window.

5. The endoscope apparatus according to claim 1, wherein:
the fluid supply channel comprises:
a flow channel of a fluid spout member that is provided in the fluid spout and in which a channel that links the fluid inlet and the fluid spout is formed in a curved shape, a groove portion channel provided in an end portion of the optical adapter, an inlet passage formed in the fluid inlet, and an adapter-side supply conduit that is formed inside the optical adapter main body.

6. The endoscope apparatus according to claim 2, wherein:
the fluid supply channel comprises:
a flow channel of a fluid spout member that is provided in the fluid spout and in which a channel that links the fluid inlet and the fluid spout is formed in a curved shape, a groove portion channel provided in an end portion of the optical adapter, an inlet passage formed in the fluid inlet, and an adapter-side supply conduit that is formed inside the optical adapter main body.

7. The endoscope apparatus according to claim 5, wherein the groove portion channel is provided in an end face that has the fluid inlet of the optical adapter.

8. The endoscope apparatus according to claim 6, wherein the groove portion channel is provided in an end face that has the fluid inlet of the optical adapter.

9. The endoscope apparatus according to claim 1, wherein:
the fluid supply channel comprises:
a first supply conduit that is linearly provided along an insertion direction through the fluid inlet, and a second supply conduit that is linearly provided along an opposite direction to the insertion direction through the fluid spout side;
wherein the first supply conduit and the second supply conduit are configured so that a portion of the first supply conduit and a portion of the second supply conduit that are out of alignment in a direction orthogonal to the insertion direction and are formed so as to overlap and communicate with one another.

10. The endoscope apparatus according to claim 2, wherein:
the fluid supply channel comprises:
a first supply conduit that is linearly provided along an insertion direction through the fluid inlet, and a second supply conduit that is linearly provided along an opposite direction to the insertion direction through the fluid spout side;
wherein the first supply conduit and the second supply conduit are configured so that a portion of the first supply conduit and a portion of the second supply conduit that are out of alignment in a direction orthogonal to the insertion direction and are formed so as to overlap and communicate with one another.

11. The endoscope apparatus according to claim 1, wherein:
the fluid supply channel comprises:
a first supply conduit that is linearly provided along an insertion direction through the fluid inlet;
a second supply conduit that communicates with the fluid spout and that is linearly provided along an opposite direction to the insertion direction;
a third supply conduit that is provided in a direction orthogonal to the insertion direction and that causes the first supply conduit and the second supply conduit to communicate; and
a fourth supply conduit that is provided in a direction orthogonal to the insertion direction, which causes a conduit that supplies fluid to the fluid spout and that is provided in an ejection portion that includes the fluid spout, and the second supply conduit to communicate.

12. The endoscope apparatus according to claim 2, wherein:
the fluid supply channel comprises:
a first supply conduit that is linearly provided along an insertion direction through the fluid inlet;
a second supply conduit that communicates with the fluid spout and that is linearly provided along an opposite direction to the insertion direction;
a third supply conduit that is provided in a direction orthogonal to the insertion direction and that causes the first supply conduit and the second supply conduit to communicate; and
a fourth supply conduit that is provided in a direction orthogonal to the insertion direction, which causes a conduit that supplies fluid to the fluid spout and that is provided in an ejection portion that includes the fluid spout, and the second supply conduit to communicate.

13. A plurality of kinds of optical adapters that can be detachably attached to a distal end portion of an endoscope insertion portion, and that comprise an illuminating window that radiates an illuminating light and an observation window through which a subject can be viewed, wherein:
a fluid spout that ejects fluid at least at the observation window, and a fluid inlet that is connected to and communicates with a fluid supply port for supplying the fluid that is arranged on a distal end face of the distal end portion of the endoscope insertion portion are provided in the optical adapter; and
a fluid supply channel that causes the fluid inlet and the fluid spout to communicate and in which the fluid flows, and the fluid spout, are configured differently according to a kind of the optical adapter.
